(19) United States
(12) Patent Application Publication
(10) Pub. No.: US 2026/0199634 A1
(43) Pub. Date: Jul. 16, 2026

(54) STERILIZABLE MAGNETIC COUPLING MECHANISM FOR KNOB-BASED CATHETER CONTROL

(71) Applicant: Siemens Medical Solutions USA, Inc., Malven, PA (US)

(72) Inventors: Young-Ho Kim, West Windsor, NJ (US); Ankur Kapoor, Plainsboro, NJ (US)

(21) Appl. No.: 19/342,919

(22) Filed: Sep. 29, 2025

Related U.S. Application Data
(60) Provisional application No. 63/744,918, filed on Jan. 14, 2025.

(51) Int. Cl. A61M 25/01; A61B 34/30

(52) U.S. Cl. CPC A61M 25/0113; A61B 2034/301; A61M 2205/0272

(57) ABSTRACT

A magnet-based coupling mechanism and an external gear system for a robotic catheter system. The magnet-based coupling mechanism and external gear system decouple knob operations of a catheter handle from body movements, allowing for precise robotic integration and sterilization.

### CROSS REFERENCE TO RELATED APPLICATIONS

This application claims the benefit of the filing date under 35 U.S.C. § 119(e) of U.S. Provisional Application Serial No. 63/744,918 filed January 14, 2025.

### FIELD

This disclosure relates to robotic medical catheters.

### BACKGROUND

Medical catheters are flexible, tube-like devices inserted into the body to deliver therapies, drain fluids, or enable diagnostic procedures. To assist in navigating the catheter, a robotic catheter navigation system may be implemented for assisted steering of the catheter to enable a user (e.g., cardiologist, a clinician, or any other user) to manipulate the catheter in all four degrees of freedom (i.e., anterior/posterior tip bending, left/right tip bending, rotation, and translation) needed to fully steer the catheter.

Robotic catheter systems enable precise navigation and manipulation of catheters within the body. Mechanically, robotic catheter systems link instructions to a robotic drive unit that interfaces with standard vascular access devices and actuates catheters, guidewires, and sheaths with high precision. The system may remove a physician from the immediate field while providing fine motor control that often exceeds what can be achieved with manual manipulation. Robotic catheter systems may further provide fully automated catheter steering.

In a typical medical procedure that uses a catheter, a catheter handle of the catheter is used to control the distal end of the catheter. Such a catheter handle typically features knobs mechanically coupled to the body (for manual operation), resulting in the movement of the entire handle during operation. This complicates precise integration with robotic systems. Additionally, ensuring sterilizability for reusable components while maintaining cost-effectiveness and simplicity for disposable parts is a significant challenge.

### SUMMARY

By way of introduction, the preferred embodiments described below include methods, systems, instructions, and/or computer readable media for a sterilizable magnetic coupling mechanism for knob-based catheter control.

In a first aspect, a robotic catheter system comprising: a catheter comprising at least a catheter handle with a set of auxiliary gears configured to control a movement, an orientation, or a positioning of the catheter; and a base section comprising: a sealed structure comprising a plurality of motors located inside the sealed structure, the plurality of motors coupled with a set of external gears located outside the sealed structure; the set of external gears configured to couple with the set of auxiliary gears of the catheter handle; and one or more mounting platforms configured to securely hold the catheter handle of the catheter, wherein one or more contact points between the catheter handle and the one or more mounting platforms use magnetic connections to securely hold the one or more mounting platforms and catheter handle, wherein the catheter handle is configured to be removed by breaking the magnetic connections.

In a second aspect, a mounting system for a robotic medical catheter, the mounting system comprising: a set of auxiliary gears coupled with one or more knobs on a catheter handle, the set of auxiliary gears configured to interact with a set of external gears of a base section; and a mounting platform connected to the base section, the mounting platform comprising one or more magnetic contact points that are configured to hold the catheter handle in a predefined location and orientation.

In a third aspect, a catheter handle of a robotic catheter system, the catheter handle comprising: a plurality of knobs configured to control a movement, an orientation, and/or a position of a distal end of the robotic catheter system; a plurality of auxiliary gears connected to the plurality of knobs, the plurality of auxiliary gears configured to interface with a set of external gears on a base section; and one or more magnetic contact points configured to magnetically secure the catheter handle to a mounting platform of the base section.

Any one or more of the aspects described above may be used alone or in combination. These and other aspects, features and advantages will become apparent from the following detailed description of preferred embodiments, which is to be read in connection with the accompanying drawings. The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale; emphasis instead being placed upon illustrating the principles of the embodiments. Moreover, in the figures, like reference numerals designate corresponding parts throughout the different views.

FIG. 1 depicts an example system for a sterilizable magnetic coupling mechanism for knob-based catheter control according to an embodiment.

FIG. 2 depicts an example mounting platform and external gears according to an embodiment.

FIG. 3 depicts example mounting platform according to an embodiment.

FIG. 4 depicts a schematic of an example catheter handle according to an embodiment.

FIG. 5 depicts a photograph of an example catheter handle according to an embodiment.

FIG. 6 depicts an example base section with mounting platforms according to an embodiment.

FIG. 7 depicts a photograph of an example base section with mounting platforms according to an embodiment.

FIG. 8 depicts an example base section, mounting platforms, and catheter handle according to an embodiment.

FIG. 9 depicts a photograph of an example base section, mounting platforms, and catheter handle according to an embodiment.

FIG. 10 depicts an example controller of the base section according to an embodiment.

### DETAILED DESCRIPTION

Embodiments described herein provide a magnet-based coupling mechanism and an external gear system to decouple knob operations from body movements, allowing for precise robotic integration. Furthermore, the design facilitates easy attachment and detachment of disposable catheters, ensures clear orientation, and incorporates a sealed structure to enable effective sterilization, addressing both usability and hygiene requirements.

The robotic catheter system described herein may fit or conform to standard medical catheters such as commercially-available intracardiac echocardiography (ICE) catheters. The embodiments described below relate to a typical steerable catheter, but other types of catheter may be used The magnet-based coupling mechanism and an external gear system of the sterile robotic system provides for full-DOF robotic control of the catheter. This system may be used for any kind of catheter or flexible medical instrument for insertion into a patient, including but not limited to bronchoscopes, flexible endoscopes, trans-thoracic echocardiography probes, trans-esophageal echocardiography probes, cardiac catheters for intervention (i.e., with medical tool for interaction with tissue of the patient), and any other type of catheter.

Where the catheter is an ICE catheter, the robotic catheter system may be configured to control the catheter, including the imaging provided by the catheter and for the movement of the catheter (for example using additional imaging modalities). In a procedure, an array of elements provided by the catheter is controlled through beamforming to generate ultrasound images. The field of view, type of imaging, and/or other imaging control may be provided. Where the catheter is an ablation or other treatment catheter, the robotic catheter system may be configured to control the ablation or treatment catheter. For example, the contact sensing and/or electrode or another ablation applicator is controlled to ablate or treat. The ablation control may be performed by the robotic catheter system.

In an example catheter based operation, a distal end of the catheter is inserted into a patient. At the proximal end of the catheter is a handle that may be typically used for control of the catheter, in particular control of the distal end of the catheter. An example imaging catheter (for example, an ICE catheter) includes an array of elements for imaging within a shaft or housing having a tip, electrical conductors, steering wires, and the handle. Additional, different, or fewer components may be provided, such as radio opaque markers, ablation electrodes, lens, needle guide, or ports. In other embodiments, the catheter is an ablation catheter or interventional catheter rather than an imaging catheter.

The shaft housing of the catheter is PEBAX, nylon, polymer, or other flexible material. The shaft housing is formed around the array of elements for imaging and other parts extending from the handle and insertable into the patient. The shaft housing is configured for insertion into a circulatory system of a patient. For example, the distal tip of the catheter includes a more flexible portion covered by the shaft housing for moving through the circulatory system. Steering wires connected to the shaft housing or parts (e.g., anchors) within the housing are configured to guide the shaft housing within the circulatory system. The array of elements for imaging is positioned within the catheter. The array of elements for imaging is at a distal end or tip of the catheter, such as being within 20 mm of an end of the tip or a beginning of a flexible tip portion. The array of elements for imaging may have any position within the catheter that results in the array being within the patient during use of the catheter for imaging. Electrical conductors connect the elements of the array to the beamformer. The conductors are cables, coaxial cables, traces on flexible circuit material, wires, wire jumpers, combinations thereof, and/or other now known or later developed conductor. The beamformer includes a plurality of channels for generating transmit waveforms and/or receiving signals. Relative delays and/or apodization focus the transmit waveforms or received signals for forming beams and setting a focal location. The beamformer connects with the conductors for applying waveforms for imaging with the array and receiving signals. For imaging, the beamformer selects an aperture including one, some, or all of the elements of the array. For scanning, the beamformer electronically focuses along the azimuth direction. A plurality of scan lines using an aperture is scanned. During receive operations, the focus may vary as a function of depth (i.e., dynamic focusing).

The steering wires of the catheter are used to position the array (and/or medical instrument) relative to the patient. The steering wires are cables, tendons, or other structure for transferring push and pull force from the handle to a portion of the catheter within a patient, such as to the distal end or the tip. Any material may be used, such as plastic, fiberglass, or metal. Any number of steering wires may be used, such as three or four wires. For example, three or four steering wires offset from the center in an equal spacing about the center or longitudinal axis may be used to steer along two perpendicular planes. The steering wires run through the elastic material of the catheter body or shaft housing to the distal end and are arranged in a circular fashion around a central channel, which provides sufficient space for the ultrasound transducer cable or conductors to be guided through. The relative force between the steering wires causes the catheter to bend. Any now known or later developed arrangement of steering wires may be used.

In a traditional catheter, knobs are provided for controlling an operation of the distal end in each plane. The steering wires of the catheter are connected to one or more knobs. Rotation of a knob causes a change in relative pressure or force on the steering wires which provides a movement or operation in one or more directions at the distal end of the catheter. More than one knob may be provided, for example two or three knobs. One knob (e.g., AP knob) may be configured for bending in one plane, such as an anterior-posterior (AP) plane, and another knob (e.g., RL knob) is configured for bending in a perpendicular plane, such as a right-left (RL) plane. In an embodiment, the knobs rotate to steer the distal end of the catheter by adjusting the steering wires. The knobs rotates about a longitudinal axis of the handle and/or catheter. For example, the knob is a cylinder or ring on the outer housing of the handle for rotation about the axis in either direction to steer. In other examples, a knob rotates around an axis perpendicular to the longitudinal axis of the handle. Alternatively, a slider or lever is provided as the knob for steering.

The steering wires control the bend at a distal end of the catheter. The bend may be at a portion of the catheter adjacent to the distal end or tip, such as providing for the array to be spaced from the handle by the bend. For example, the steering wires are anchored to the shaft housing, transducer array, or a rigid insert or anchor near the distal end to cause the bend. The elastic body or shaft housing may be bent along its principal axes by applying tension to the attached steering wires. Using motors instead of user-based rotation of two knobs for two planes may allow for only three steering wires for forming the bend. Four steering wires with motor-based control may be used, such as where the handle is also designed for manual operation.

A typical handle of a catheter includes a housing and user input in the form of the one or more knobs. The handle is shaped and sized for handheld guidance or use of the catheter. For example, the handle is cylindrical with grips to be used by one hand of a surgeon. The handle has a single housing made of one or more parts. The housing connects with the shaft housing of the catheter and with a cable or cables for power and communications. In embodiments described herein, the housing and handle are shaped and configured for use with a robotic system rather than or in addition to handheld guidance.

Movement of the knobs of traditional catheter handles typically result in a movement of the entire handle during operation, which complicates precise integration with robotic systems. For example, an operator that turns or twists a knob may inadvertently move the entire handle in one or more directions thus throwing off any integration with a robotic control system that requires precise alignment of the catheter system. Additionally, ensuring sterilizability for reusable components while maintaining cost-effectiveness and simplicity for disposable parts is a significant challenge. This problem has traditionally been addressed using bulky conversion mechanisms that act as intermediaries between catheter handles and robotic systems. These add-on devices, while functional, often increase the system's complexity, reduce usability, and compromise sterilizability. Some approaches have attempted to redesign catheter handles entirely, creating integrated, single-unit devices specifically tailored for robotic control. However, such designs typically lack modularity, are not cost-effective for disposable use, and may require extensive re-engineering of existing systems. Despite these efforts, current solutions still face challenges in achieving precise control, seamless integration, and practical sterilization, leaving significant room for improvement.

Embodiments described herein provide an innovative integration of magnet-based coupling, external gear systems, and a modular design, that collectively address the limitations of current catheter handle and robotic system integrations. These differences offer several key advantages including magnet-based coupling, an external gearing system for knob control, a sealed and sterilizable base design, a modular configuration, and smooth rotational mechanics. For example, the magnet-based coupling mechanism allows for quick, intuitive, and secure attachment/detachment, with clear top-bottom orientation, improving user-friendliness and reducing procedural setup time. It also ensures compatibility with disposable catheter designs while maintaining reusability for other components. The external gear system for knob control enables precise decoupled control of individual knobs, allowing the robotic system to manipulate them independently without influencing the body. This enhances accuracy and smooth operation during procedures. The sealed and sterilizable base design ensures hygiene and regulatory compliance, particularly in sterile environments, while reducing operational costs by combining disposable and reusable components effectively. The scalable and modular configuration supports flexible customization, enabling scalability to more complex procedures while maintaining a compact and efficient setup. The smooth rotational mechanism with integrated magnets ensures smooth and frictionless motion, improving durability and reducing maintenance needs.

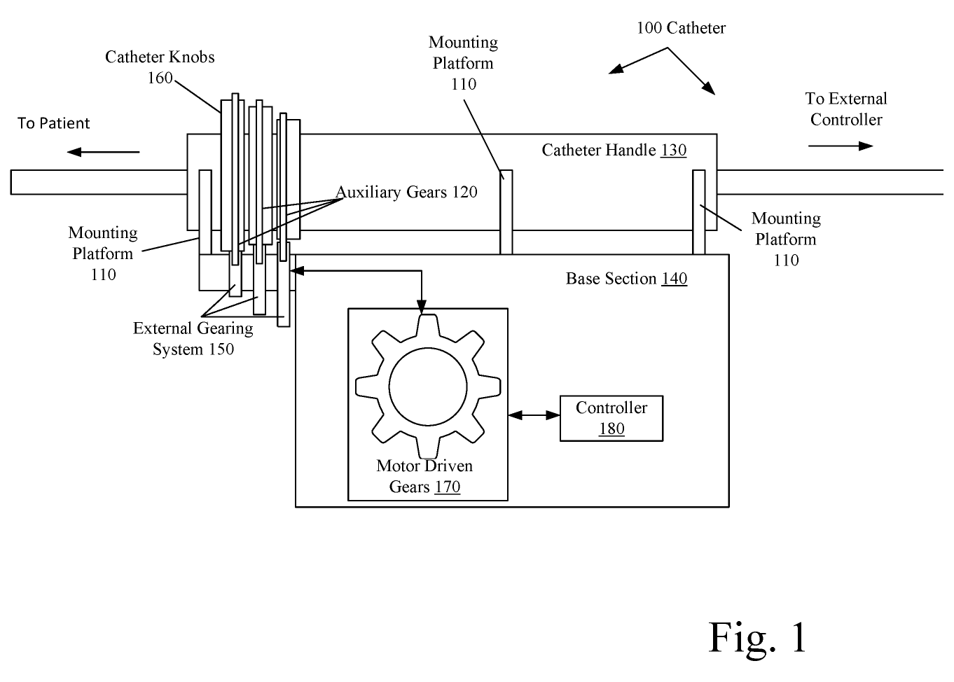

FIG. 1 depicts an example of a robotic catheter system including a catheter 100 and a sterilizable magnetic coupling mechanism for knob-based catheter control. FIG. 1 includes at least a base section 140 and one or more mounting platforms 110 that are configured to hold a catheter handle 130 in a precise alignment. The entirety of the catheter 100 is not depicted in FIG. 1, only the portion that interacts with the base section 140. Each of the components may include a housing or other covering. The housings are plastic, metal, resin, silicone, or other material. The shafts and gearing are plastic, metal, or another material. Additional component may be provided such as electrical cables for power and communications.

The base section 140 includes a controller 180 and houses motors and motor drivers (motor driven gears 170) for robotic control of the catheter, with all actuation mechanisms oriented forward. These motors are responsible for driving the external gear system 150 for precise control of the catheter. The knobs 160 which control the operation of the catheter are directly connected to the external gearing system 150 which is connected to the motor driven gears 170. Motion from the motor driven gears 170 is transferred to the external gears 150 of the catheter handle 130 which are also connected to the knobs 160. In this way, the controller 180 of the base section 140 can emulate or reproduce a procedure performed previously with movement of the knobs 160. In an embodiment, the base section 140 includes a sealed structure and a set of external gears 150, the sealed structure including a plurality of motor driven gears 170 coupled with the set of external gears 150. The seal of the base section 140 may be provided by any material that provides a sterilizable seal.

The term “sterilizable” refers to the ability of the base section 140 or other component to withstand a validated sterilization process while maintaining its safety, function, and structural integrity. For example, the base section 140 is sterilizable as the sterilization process can be validated to achieve a required sterility assurance level (SAL) by regulations such as 10⁻⁶, meaning there is less than a one-in-a-million chance of a viable microorganism remaining after sterilization. The base section 140 and other components are configured so that they can reliably undergo sterilization such as steam autoclaving, ethylene oxide treatment, gamma irradiation, low-temperature plasma sterilization, or other sterilization technique without being damaged or rendered unsafe. The base section 140 may be reusable because it can endure repeated sterilization cycles without being damaged or rendered unsafe.

The base section 140 is coupled with one or more mounting platforms 110. The mounting platforms 110 are configured to securely hold the catheter handle 130 of the catheter. Contact points between the catheter handle 130 and the mounting platforms 110 use magnetic connections to securely connect the mounting platform 110 and catheter handle 130. Magnets are placed on the mounting platform 110 or the catheter handle 130 in order to react with ferromagnetic material on the other respective component. A set of auxiliary gears 120 coupled with the knobs 160 of the catheter handle 130 are aligned with the set of external gears 150 of the base using the contact points 112 in order to interface the catheter to the motor driven gears 170 in the base section 140.

In an embodiment, the base section 140 includes the motor driven gears 170 for the adjustable degrees of freedom for control of the handle. The base contains multiple actuators, for example electric motors with high precision gearing and torque control. The catheter is secured with a mechanism (described in more detail below) that can translate and rotate the shaft with micrometer resolution. Translation may be accomplished by linear actuators / motor driven gears 170 in the base section 140 that advance or retract the catheter along its axis. Rotation is provided by rotary actuators / motor driven gears 170 that twist the catheter shaft while maintaining grip. In some systems, additional actuators control deflection at the distal tip of the catheter. The distal tip may be steerable via pull wires embedded within the catheter walls. The wires run the length of the catheter and terminate at the proximal end, where the robotic unit pulls or releases them. Pulling one wire bends the tip in one direction, while coordinated pulling of multiple wires produces complex bending maneuvers in multiple planes.

The base includes the motor driven gears 170 to manipulate the catheter in any number of degrees of freedom, such as four (4) or more degrees of freedom (e.g., global translation, global rotation, and bending or steering the catheter in two or more directions or planes). Gearing, motors, grippers (clamps), connectors, and/or other robotic components are included the base for generating and transmitting force to operate the catheter. The robotic catheter system applies push and/or pull forces to steering wires (e.g., three or four steering wires or TDCM per catheter) to operate the catheter. The tips are steered for catheter operation. Electrical signals, translation, and/or rotation force may be generated in the base. Global translation and/or rotation forces may similarly be applied, such as through the handles of the catheter.

The base section 140 may be configured as a general purpose system in that any standard device that can be fitted with an appropriate transmission system may be used with the base. The adjustable degrees of freedom and compactness of the base section 140 allow for surgeries at different access points (e.g., femoral, radial, or jugular) and precise alignment of the medical instrument, such as alignment of an ICE catheter with a sheath. The base may be easily attached to the operating table or to another mobile table using one or more fasteners or connectors. Gravity, suction cups, latches, and/or other attachment devices may be used. Larger height, width, and/or length may be used. The base section 140 may be rotated or tilted. This pose-ability of the base allows for precise alignment of the catheter with a sheath to be inserted in the patient. The sealing of the base section 140 and the mounting platform 110 between the base and the catheter handle 130 further allows for the base to remain sterile so that the entire base is more easily cleanable and thus reusable.

The base section 140 includes a plurality of motor driven gears 170 and at least a controller 180 that is configured to operate the motor driven gears 170 which are coupled to the external gears 150 which are coupled to auxiliary gears 120 on the catheter handle coupled to the knobs 160 of the catheter handle that are used to control steering wires that control the operation (movement) of the catheter.

As depicted in FIG. 1, a front (facing towards the patient) section of the base contains exposed motor-driven gears 150 that extend outward from the base section 140. These gears are precisely aligned to engage with the auxiliary gears 120 that are coupled with the knobs 160 on the catheter handle 130, enabling direct actuation of the knobs 160. The positioning ensures efficient transmission of motion without requiring additional mechanisms, simplifying the overall system design. As depicted in FIG. 1, there are three external gears 150. Additional gears 150 may be provided. In an alternative embodiment, fewer than the three gears 150 may be used for operation of the robotic catheter system. For example, only two gears 150 may be engaged with the catheter handle 130 if only a limited DOF is required. In an embodiment, the external gearing system 150 may be adjustable up or down, left or right, for example step wise, in order to interact with the respective auxiliary gears 120 on the catheter handle 130.

The exposed motor-driven gears 150 are connected to motors / motor-driven gears 170 respectively. The motors are in the base section 140, such as within the sealed metal or plastic housing of the base section 140. The motor shafts connect through gearing, such as bevel gears, to shafts. The shafts includes bevel gears that mate with the gearing. Rotation by the motor causes the shaft to rotate, which rotates the respective gear. The mating of the various gearings provides a transmission of force from the motors to the rotation and translation gears. The motors may be servo motors, rotational motors, linear motors (e.g., linear magnetic motors), or other electric, pneumatic, or hydraulic motors for rotating the shafts. Alternatively, gearing, clutch, and/or transmission is used to apply force from one motor to multiple shafts. In order to keep the electronics sterile, the motors are spaced away from the catheter by being positioned in the sealed base section 140. The exposed gears 150 are connected to gear shafts that pass through a barrier / sterilizable seal (e.g., gasket, O-ring, or cover) and into the base section where they interface with the motors 170.

The shaft passing through the barrier must be sealed in a way that both maintains sterility and allows reliable torque transfer. Different sterilizable shaft seal mechanisms may be used. A mechanical face seal may be used, with a stationary sealing ring mounted to the barrier wall and a rotating ring fixed to the shaft. The sealing interface is kept closed by a spring or bellows. Materials such as silicon carbide, ceramic, or stainless steel against polytetrafluoroethylene (PTFE), PEEK, or perfluoroelastomers (FFKMs) repeated sterilization. A bellows seal provides a hermetic metal bellows welded to the barrier, with the bellows carrying a sealing face that contacts a counter face on the shaft. The bellows accommodates shaft movement while preventing leakage. Stainless steel or Inconel bellows are standard for steam or plasma sterilization. A lip seal formed from PTFE or perfluoroelastomer may be press-fitted into the barrier wall, the flexible lip conforming around the shaft. This provides compact sealing for shafts with moderate rotation speeds and can survive autoclave cycles if engineered with proper material choice. A magnetic fluid seal may be used, where ferrofluid held in place by an annular magnetic field bridges the gap between the rotating shaft and housing. A labyrinth seal combined with a sterile purge gas may also be applied. The labyrinth geometry impedes flow along the shaft, and a sterile gas (steam or filtered air) pressurizes the seal area to prevent contamination ingress or egress. Alternatively, a magnetic coupling may be used that eliminates shaft penetration entirely, with the internal motor magnetically driving external gears across the barrier.

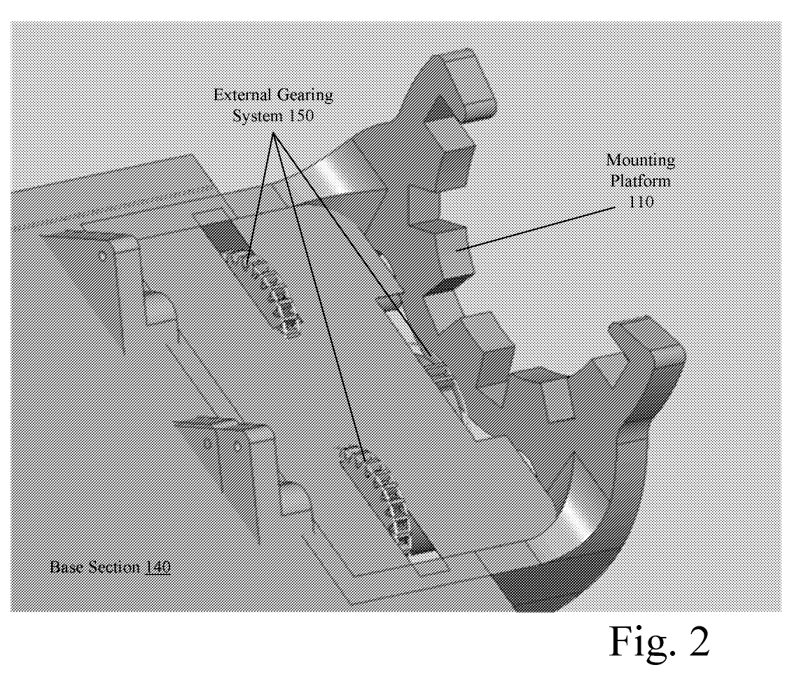

FIG. 2 depicts an example of the base section 140, a mounting platform 110 and the exposed gears 150. The mounting platform 110 (shown without magnetic connections) is configured to hold the catheter handle 130 securely and precisely in place so that auxiliary gears 120 of the catheter handle 130 are aligned with the exposed motor-driven gears 150. As depicted, the exposed motor-driven gears 150 are offset so that each is positioned to be in contact with a respective auxiliary gear of the catheter handle 130. The motor-driven gears 150 may be different types of gears 150 as depicted in FIG. 2 depending on the type of motion they are intended to convey to the auxiliary gearing system of the catheter handle 130.

The system of FIG. 1 includes one or more mounting platforms 110 that hold the catheter in place. These mounting platforms 110, e.g., the parts located on the upper part of the base section 140 are configured to securely hold the catheter handle 130 using, for example, magnetic connections. The mounting platforms 110 provide secure gripping without damaging the handle, while also enabling precise translation and rotation. In an embodiment, the contact points 112 between the catheter handle 130 and the mounting platform 110 use a plurality of magnets, allowing for easy attachment and detachment while maintaining a firm and precise alignment. The magnets may be part of the catheter handle 130 or mounting platform 110. The number and strength of the magnets may be altered to increase ease of removability or strength of the bond. The magnetic break-away function is highly suited to medical environments protecting patients, hospital staff and equipment from harm.

In an embodiment, the magnetic connections are provided by electromagnets. The electromagnets may be turned on or off by the controller 180 or other switch, such as a catheter controller 180. The electromagnets may be electro-permanent magnets. In another embodiment, the magnetic connections are provided using magnet tape. In such an embodiment, the catheter handle 130 may be wrapped in magnet tape which magnetically coupled with ferromagnetic surfaces, such as steel, iron, nickel, and cobalt that are embedded in or part of the mounting platform 110. In another embodiment, the magnetic connections are magnetic pogo-Pin Connectors. In yet another embodiment, the magnetic connections use a mechanical assist. The magnets draw and align the handle and mounting platform 110 together, while a mechanical feature (lip, hook, or latch) resists shear force.

In the example of FIG. 1, there are three mounting platforms 110. In an embodiment, only two of the three mounting platforms 110 are configured with magnetic connections. The third mounting platform 110 may be used for alignment and balance but otherwise may constrain the movement of the catheter handle 130. Additional mounting platforms 110 may be used for support or alignment. In another embodiment, each of the mounting platforms 110 includes magnetic connection, but the catheter handle 130 is only configured to magnetically connect with fewer than all of the mounting platforms 110. In such an example, the magnetic connections are present in the mounting platforms 110, but the associated contact points 112 on the catheter handle 130 are not magnetic. Fewer magnetic connections may allow for easier disengagement of the catheter handle 130 while more magnetic connections may provide for a stronger bond.

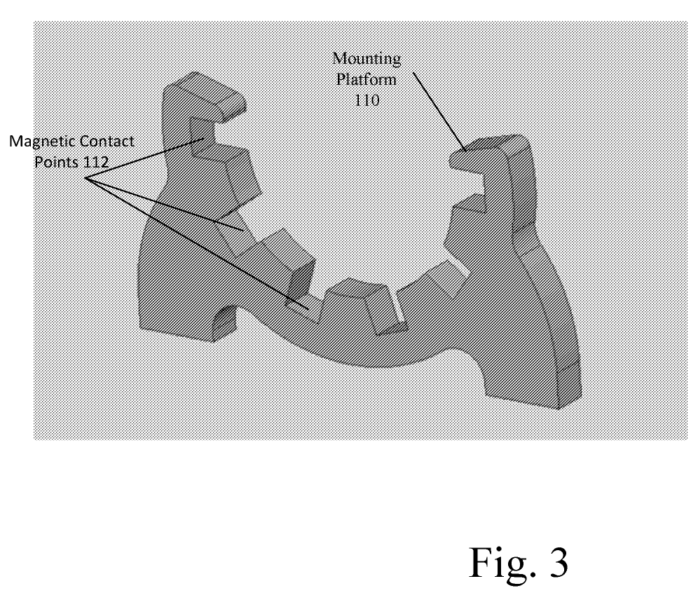

FIG. 3 depicts an example of a mounting platform 110. The mounting platform 110 is configured in the shape of a semicylinder in order to hold the cylindrical shape of the catheter handle 130. The rectangular / square voids are where magnetic contract points 112 are integrated into the mounting platform 110. In FIG. 3, there are six places where a magnet or other connector may be used. The magnets may be aligned with respective connection points on the catheter handle 130 so that the precise alignment / orientation of the catheter handle 130 and thus auxiliary gears 120 is set. Alternatively, these areas may be filled with ferromagnetic surfaces, such as steel, iron, nickel, and cobalt that magnetically attract magnetic substances on the catheter handle 130. The diameter of the semicylinder may be designed to fit particular catheter handles 130. In an embodiment, different mounting platforms 110 may be used at different locations on the base section 140 in order to comply with different types of catheters. Different shapes of catheter and/or different types of knobs 160 may require different mounting locations.

The mounting platforms 110 are configured to align the auxiliary gears 120 and the external gear system so that the two interface seamlessly. The mounting platforms 110 are further designed to securely hold the catheter handle 130 so that the catheter handle 130 does not slip during operation. In an embodiment, the mounting area features two or more magnets (or magnetic areas) strategically placed to ensure smooth rotational motion of the attached device. The magnets provide an appropriate level of holding force, allowing the device to be easily attached and detached while maintaining secure alignment. In an example, the mounting points include one at the front and one at the middle section, ensuring stable support and balance for the device.

Referring back to FIG. 1, the system may include a catheter handle 130 that is configured to interface with the mounting platforms 110 and the exposed motor-driven gears. The handle 130 includes a housing and user input in the form of one or more knobs 160. The handle 130 is shaped and sized for handheld guidance or use of the catheter. For example, the handle 130 is cylindrical with grips to be used by one hand of a surgeon. The handle 130 has a single housing made of one or more parts. The housing connects with the shaft housing of the catheter and with a cable or cables for power and communications.

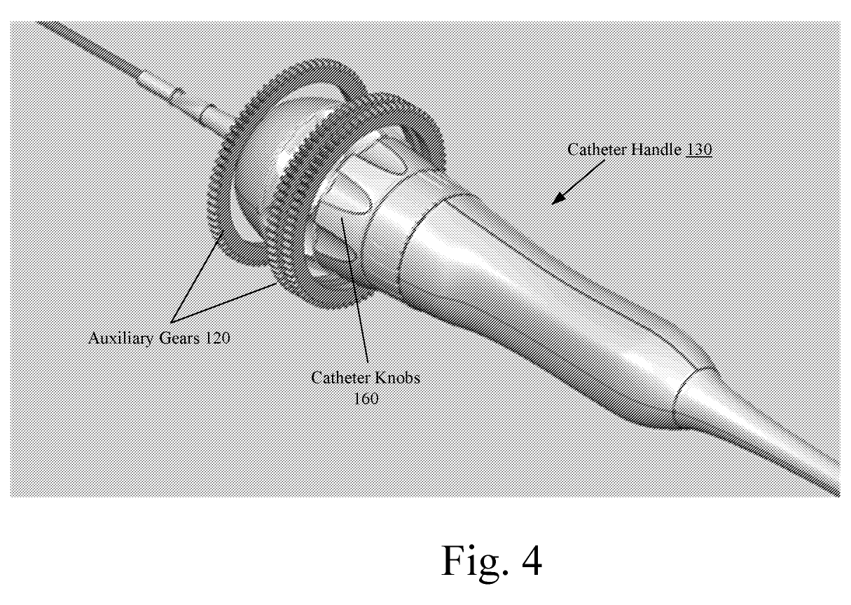

FIG. 4 depicts an example schematic of a catheter handle 130. The catheter handle 130 includes a plurality of knobs 160 that interact with a plurality of steering wires (not shown but located inside the catheter handle 130) that control the movement, orientation, and positioning of the distal end of the catheter. Additional knobs 160 or controls may provide control over imaging or other features of the catheter, for example used in an ablation procedure. A plurality of auxiliary gears 120 interface with the plurality of knobs 160 so that when a gear of the auxiliary gears 120 turns, the respective knob also rotates in a similar manner. The auxiliary gears 120 and knobs 160 may be connected by any means, for example using friction, tension, fasteners, connectors, or other materials. The auxiliary gears 120 may be permanently affixed to the knobs 160 or may be removable. The auxiliary gears 120 may be formed to fit dimples or pads of the knobs 160 so that the gears and knobs 160 do not slip against one another.

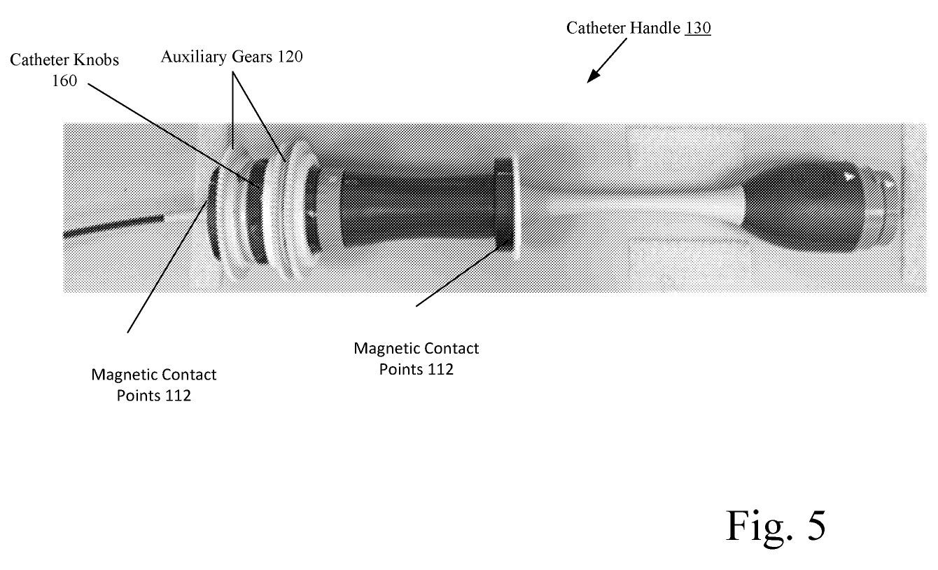

FIG. 5 depicts a photograph of a catheter handle 130 that is similar to the schematic of FIG. 4. The catheter handle 130 of FIG. 5 includes one or more sections (not shown in FIG. 3) that are configured to magnetically connect to the mounting platform 110, for example using magnetic tape. These sections may be metallic or otherwise magnetic. These sections may be removeable similar to the auxiliary gears 120 so that a standard catheter handle 130 may be converted into a catheter handle 130 that can interact with the system described herein.

The catheter handle 130 is configured to provide automatic control of the catheter through the connection with the motors and gears of the base section 140. The catheter handle 130 provides for control with one or more knobs 160 that are directly connected to the motors and gears. In an embodiment, the device features cylindrical knobs 160 centered around the body of the catheter handle 130. Auxiliary gears 120 are attached to the outer surfaces of these knobs 160, allowing them to interface directly with the motor-driven gears on the base system. The auxiliary gears 120 may be form fitted to attach to the knobs 160. The attachment to the knobs 160 may be connected by any secure means. The auxiliary gears 120 enable smooth transmission of motion for precise control of the catheter. Manual control may thus be replicated by the motor driven gears 170 and vice versa. One or more knobs 160 may be used. For example, the first (front-most) knob is responsible for controlling the rotation of the entire device body. The middle section of the handle includes two knobs 160 that are used for directional control, including Up/Down and Left/Right motions. The design also supports scalability, allowing for the addition of two or more knobs 160 with the same format for extended functionality.

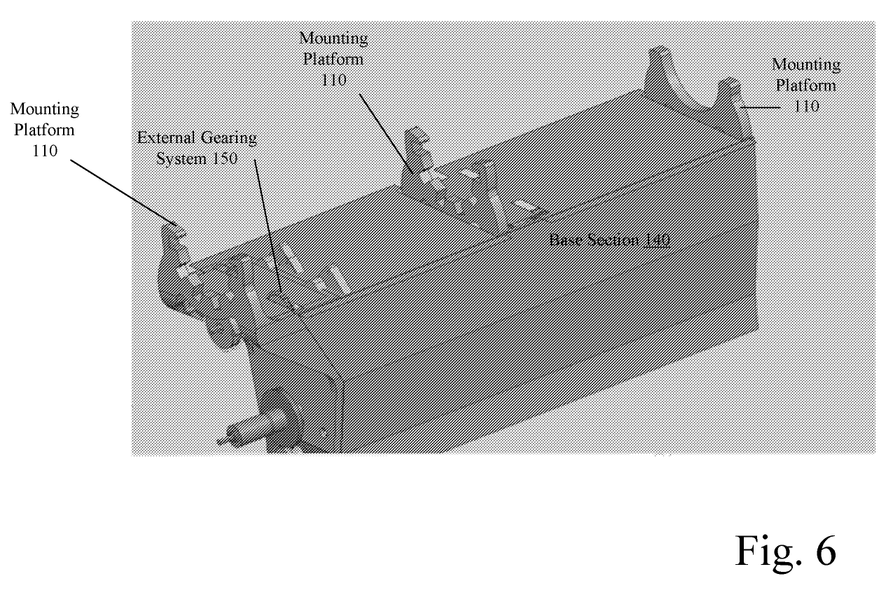

FIG. 6 depicts an example schematic of the base section 140 and the mounting platforms 110. FIG. 6 depicts three mounting platforms 110, two of which are configured for magnetic inserts which provide contact points 112 for securing the catheter handle 130 (not shown). The third mounting platform 110 may be used for alignment and support of the handle. In FIG. 6, the external gears 150 of the base section 140 are also depicted. The external gears 150 are outside of the base section 140 in order to allow for easy cleaning / sterilization without affecting the motors / controller 180 inside the sealed base section 140.

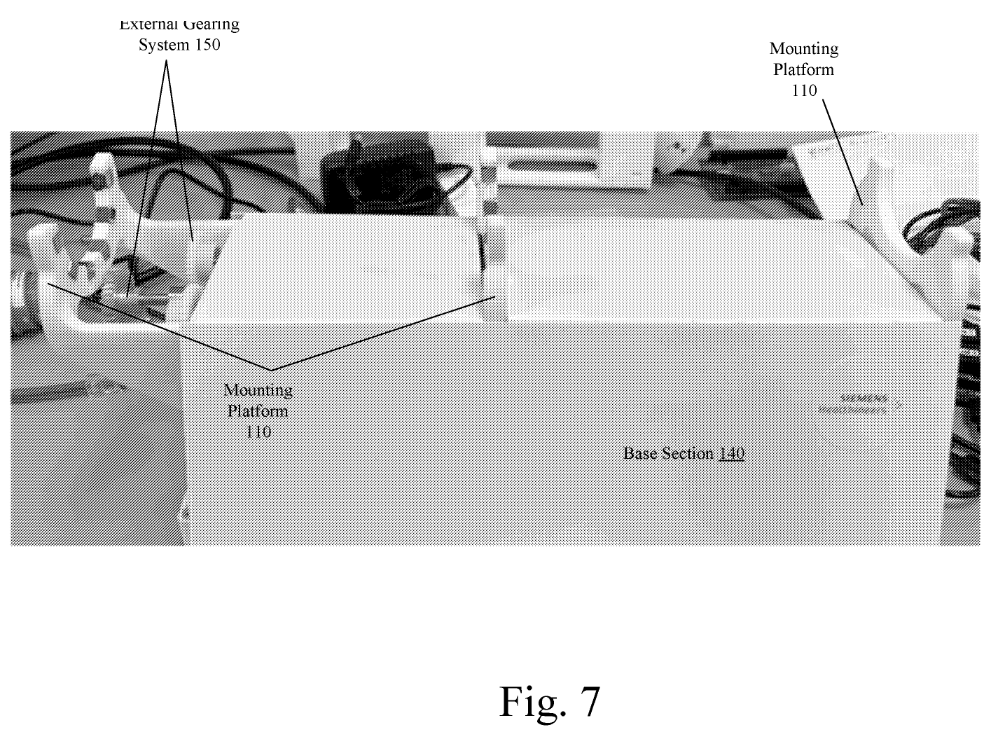

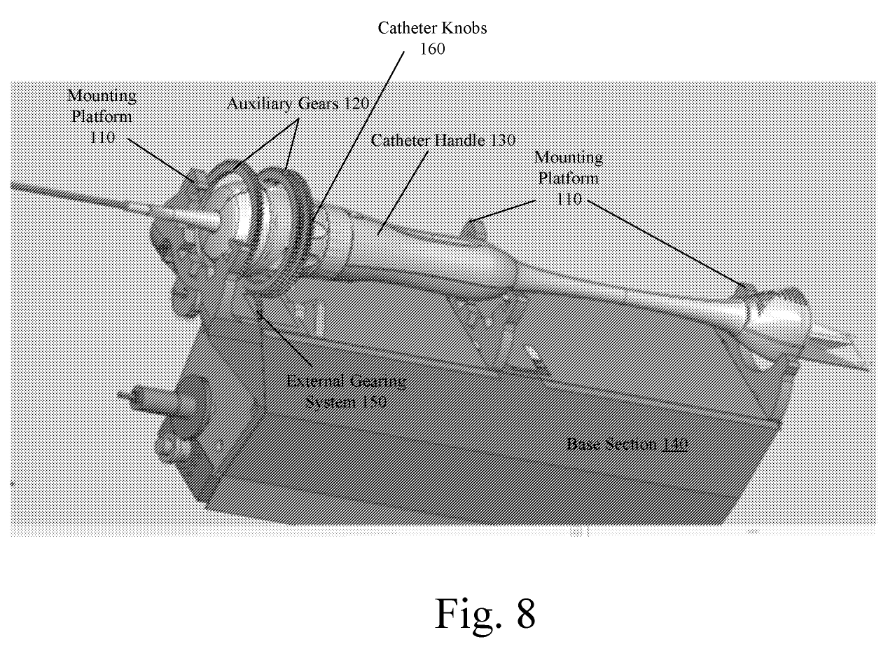

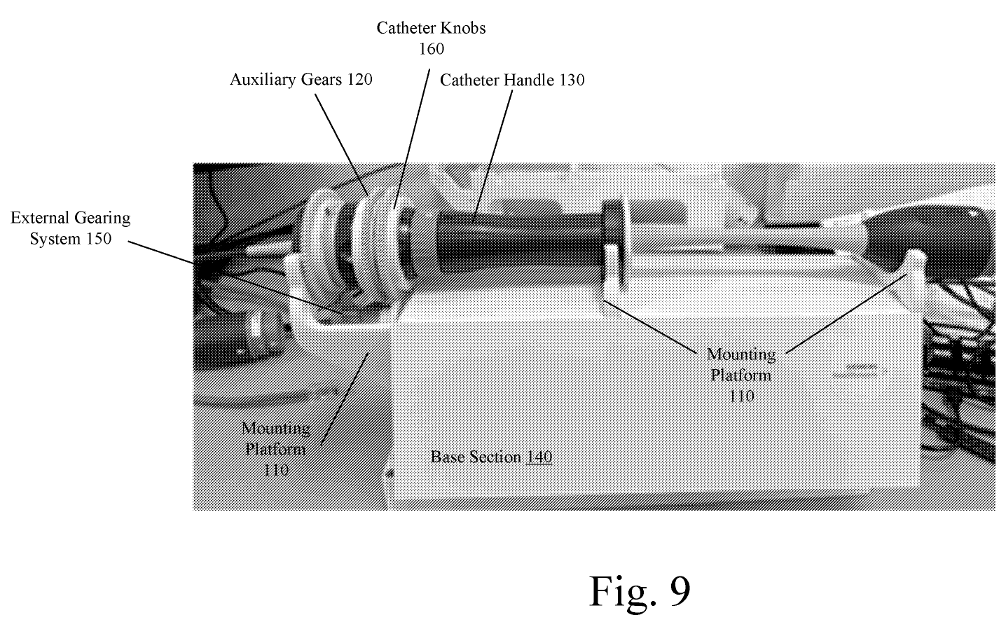

FIG. 7 depicts an example photograph of the base section 140 and the mounting platforms 110. Similar to FIG. 6, FIG. 7 depicts the base section 140 including the external gearing and three mounting platforms 110. FIG. 7 further depicts magnets or magnetic material used as contact points 112 for securing the catheter handle 130. FIG. 8 depicts an example schematic of a catheter handle 130 connected to the base section 140 via the mounting platforms 110. The auxiliary gears 120 connected to the catheter knobs 160 interface with the external gearing system 150. FIG. 9 depicts an example photograph of a catheter handle 130 connected to the base section 140 via the mounting platforms 110. The system in FIG. 9 is similar to that of FIG. 8. The mounting platforms 110 align the catheter handle 130 so that the auxiliary gears 120 connected to the catheter knobs 160 interface with the external gearing system 150.

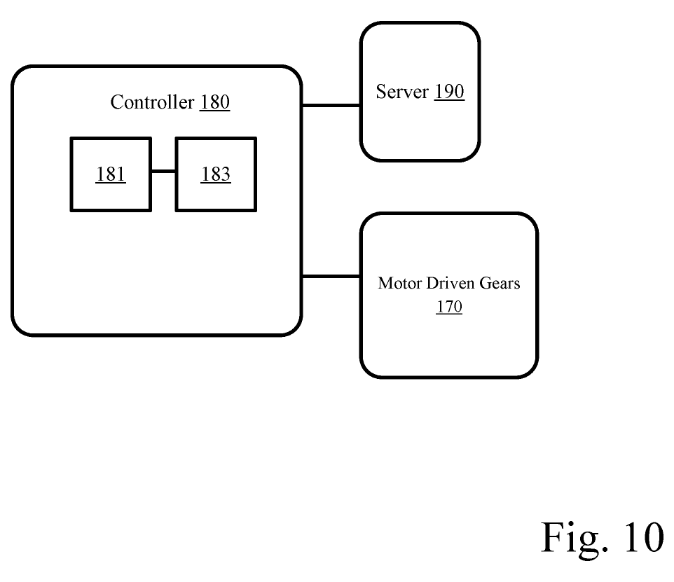

Referring back to FIG. 1, the robotic catheter navigation system further includes a controller 180. For robotic guidance, the catheter handle 130 is placed on the base section 140 and secured by the mounting platform 110. Rather than handheld use, the base section 140 provides control for rotation, steering, and/or translation via the handle to be automated. The operator is then able to tele-operate the catheter from a remote console, or a high-level motion planning algorithm may be used to generate spatial trajectories for the catheter tip. The controller 180 controls the robotic guidance. FIG. 10 depicts an example controller 180 that is configured to operate the robotic catheter system, in particular the operation of the motor driven gears 170. The controller 180 is a general processor 181, digital signal processor 181, graphics processing unit, application specific integrated circuit, field programmable gate array, artificial intelligence processor 181, digital circuit, analog circuit, combinations thereof, or other now known or later developed device for controlling the motor driven gears 170 and the movement, orientation, and/or position of the catheter, among other processes described below. The controller 180 is a single device, a plurality of devices, or a network. For more than one device, parallel or sequential division of processing may be used. Different devices making up the controller 180 may perform different functions. The controller 180 operates pursuant to stored instructions to perform various acts described herein. The controller 180 is configured by design, hardware, and/or software to translate and/or rotate the shaft using control from a user interface or input or other controls. The controller 180 is configured to control operation of the gears, thus controlling the translation and the rotation. In an embodiment, the controller 180 is in communication with a server 190 or other computing device that provides instructions and/or feedback to the controller 180 of the base section 140 in order to provide control of the robotic catheter. The controller 180 further includes memory 183 which may be or include an external storage device, RAM, ROM, database, and/or a local memory (e.g., solid state drive or hard drive). The memory 183 stores instructions for operating the robotic catheter system. The memory 183 may include computer readable storage media including various types of volatile and nonvolatile storage media. The functions, acts or tasks illustrated in the figures or described herein are executed in response to one or more sets of instructions stored in or on computer readable storage media. The functions, acts or tasks are independent of the instructions set, storage media, controller 180, or processing strategy and may be performed by software, hardware, integrated circuits, firmware, micro code, and the like, operating alone or in combination. In one embodiment, the instructions are stored on a removable media device for reading by local or remote systems. In other embodiments, the instructions are stored in a remote location for transfer through a computer network. In yet other embodiments, the instructions are stored within a given computer, CPU, GPU, or system. The server 190 may perform similar functions as the processor 181 and memory 183 but may not be co-located at the hospital / healthcare site. The server 190 may be configured in the cloud and/or used specialize hardware and software.

In operation, during and/or after steering and/or positioning, the catheter is used. For an intervention catheter, drugs may be injected from the catheter or a tool on the catheter is used (e.g., scissors, needle, ablation electrode, scalpel, or another instrument). For an imaging catheter, a transducer is used for ultrasound scanning in a field of view. Ultrasound imaging is performed with the transducer. The user may view the surrounding tissue in different directions by rotating the catheter and/or other steering. Changes in bending may alter the field of view to image other anatomy or devices in the patient. After use of the catheter, the catheter handle 130 may be removed by using enough force to break the magnetic connection or, for example, turning off electromagnets. The catheter and/or base section 140 may be cleaned, sterilized, and reused.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description. Independent of the grammatical term usage, individuals with male, female or other gender identities are included within the term.

The following is a list of non-limiting illustrative embodiments disclosed herein: Illustrative embodiment 1: A robotic catheter system comprising: a catheter comprising at least a catheter handle with a set of auxiliary gears configured to control a movement, an orientation, or a positioning of the catheter; and a base section comprising: a sealed structure comprising a plurality of motors located inside the sealed structure, the plurality of motors coupled with a set of external gears located outside the sealed structurel the set of external gears configured to couple with the set of auxiliary gears of the catheter handle; and one or more mounting platforms configured to securely hold the catheter handle of the catheter, wherein one or more contact points between the catheter handle and the one or more mounting platform use magnetic connections to securely hold the one or more mounting platforms and catheter handle, wherein the catheter handle is configured to be removed by breaking the magnetic connections.

Illustrative embodiment 2. The robotic catheter system of a previous illustrative embodiment, wherein at least one of the one or more mounting platforms is located at a section of the base section outside the set of external gears.

Illustrative embodiment 3. The robotic catheter system of a previous illustrative embodiment, wherein the one or more contacts points of the one or more mounting platforms each comprise a plurality of electromagnets to hold the catheter handle.

Illustrative embodiment 4. The robotic catheter system of a previous illustrative embodiment, wherein the one or more mounting platforms are configured as a semicylinder.

Illustrative embodiment 5. The robotic catheter system of a previous illustrative embodiment, wherein the catheter handle is equipped with magnet tape in order to secure hold the one or more mounting platforms to the catheter handle.

Illustrative embodiment 6. The robotic catheter system of a previous illustrative embodiment, wherein the set of external gears comprises three or more gears.

Illustrative embodiment 7. The robotic catheter system of a previous illustrative embodiment, wherein the catheter handle comprises one or more knobs coupled with the set of auxiliary gears, wherein the one or more knobs are rotated to control a movement, orientation, or positioning of the catheter.

Illustrative embodiment 8. The robotic catheter system of a previous illustrative embodiment, wherein a first knob of the one or more knobs is responsible for controlling a rotation of the catheter, wherein a second knob and third knob of the one or more knobs are used for directional control including Up/Down and Left/Right motions.

Illustrative embodiment 9. The robotic catheter system of a previous illustrative embodiment, wherein the base section comprises: a first motor configured by a first gearing to rotate the catheter about a longitudinal axis; and a second motor configured by a second gearing to rotate a first knob of the one or more knobs.

Illustrative embodiment 10. A mounting system for a robotic medical catheter, the mounting system comprising: a set of auxiliary gears coupled with one or more knobs on a catheter handle, the set of auxiliary gears configured to interact with a set of external gears of a base section; and a mounting platform connected to the base section, the mounting platform comprising one or more magnetic contact points that are configured to hold the catheter handle in a predefined location and orientation.

Illustrative embodiment 11. The mounting system of a previous illustrative embodiment, wherein the catheter handle includes magnetic tape that is magnetically coupled to the one or more magnetic contact points of the mounting platform.

Illustrative embodiment 12. The mounting system of a previous illustrative embodiment, wherein the base section is a sterilizable device validated to achieve a threshold sterility assurance level.

Illustrative embodiment 13. The mounting system of a previous illustrative embodiment, wherein the set of auxiliary gears are coupled with three knobs on the catheter handle, wherein a first knob is responsible for controlling a rotation of the robotic medical catheter, wherein a second knob and third knob are used for directional control.

Illustrative embodiment 14. The mounting system of a previous illustrative embodiment, wherein the one or more magnetic contact points are electromagnetic.

Illustrative embodiment 15. The mounting system of a previous illustrative embodiment, wherein the one or more magnetic contact points are electro-permanent.

Illustrative embodiment 16. The mounting system of a previous illustrative embodiment, wherein the mounting platform is composed of sterilizable materials.

Illustrative embodiment 17. A catheter handle of a robotic catheter system, the catheter handle comprising: a plurality of knobs configured to control a movement, an orientation, and/or a position of a distal end of the robotic catheter system; a plurality of auxiliary gears connected to the plurality of knobs, the plurality of auxiliary gears configured to interface with a set of external gears on a base section; and one or more magnetic contact points configured to magnetically secure the catheter handle to a mounting platform of the base section.

Illustrative embodiment 18. The catheter handle of a previous illustrative embodiment, wherein a first knob of the plurality of knobs is responsible for controlling a rotation of the robotic catheter system, wherein a second knob and third knob of the plurality of knobs are used for directional control.

Illustrative embodiment 19. The catheter handle of a previous illustrative embodiment, wherein the one or more magnetic contact points are provided by magnetic tape on the catheter handle.

Illustrative embodiment 20. The catheter handle of a previous illustrative embodiment, wherein the one or more magnetic contact points are provided by electromagnets embedded in the mounting platform.

1. A robotic catheter system comprising:
a catheter comprising at least a catheter handle with a set of auxiliary gears configured to control a movement, an orientation, or a positioning of the catheter; and a base section comprising:
a sealed structure comprising a plurality of motors located inside the sealed structure, the plurality of motors coupled with a set of external gears located outside the sealed structure;
the set of external gears configured to couple with the set of auxiliary gears of the catheter handle; and
one or more mounting platforms configured to securely hold the catheter handle of the catheter, wherein one or more contact points between the catheter handle and the one or more mounting platform use magnetic connections to securely hold the one or more mounting platforms and catheter handle, wherein the catheter handle is configured to be removed by breaking the magnetic connections.

2. The robotic catheter system of claim 1, wherein at least one of the one or more mounting platforms is located at a section of the base section outside the set of external gears.

3. The robotic catheter system of claim 1, wherein the one or more contacts points of the one or more mounting platforms each comprise a plurality of electromagnets to hold the catheter handle.

4. The robotic catheter system of claim 1, wherein the one or more mounting platforms are configured as a semicylinder.

5. The robotic catheter system of claim 1, wherein the catheter handle is equipped with magnet tape in order to secure hold the one or more mounting platforms to the catheter handle.

6. The robotic catheter system of claim 1, wherein the set of external gears comprises three or more gears.

7. The robotic catheter system of claim 1, wherein the catheter handle comprises one or more knobs coupled with the set of auxiliary gears, wherein the one or more knobs are rotated to control a movement, orientation, or positioning of the catheter.

8. The robotic catheter system of claim 7, wherein a first knob of the one or more knobs is responsible for controlling a rotation of the catheter, wherein a second knob and third knob of the one or more knobs are used for directional control including Up/Down and Left/Right motions.

9. The robotic catheter system of claim 7, wherein the base section comprises:
a first motor configured by a first gearing to rotate the catheter about a longitudinal axis; and
a second motor configured by a second gearing to rotate a first knob of the one or more knobs.

10. A mounting system for a robotic medical catheter, the mounting system comprising:
a set of auxiliary gears coupled with one or more knobs on a catheter handle, the set of auxiliary gears configured to interact with a set of external gears of a base section; and
a mounting platform connected to the base section, the mounting platform comprising one or more magnetic contact points that are configured to hold the catheter handle in a predefined location and orientation.

11. The mounting system of claim 10, wherein the catheter handle includes magnetic tape that is magnetically coupled to the one or more magnetic contact points of the mounting platform.

12. The mounting system of claim 10, wherein the base section is a sterilizable device validated to achieve a threshold sterility assurance level.

13. The mounting system of claim 10, wherein the set of auxiliary gears are coupled with three knobs on the catheter handle, wherein a first knob is responsible for controlling a rotation of the robotic medical catheter, wherein a second knob and third knob are used for directional control.

14. The mounting system of claim 10, wherein the one or more magnetic contact points are electromagnetic.

15. The mounting system of claim 10, wherein the one or more magnetic contact points are electro-permanent.

16. The mounting system of claim 10, wherein the mounting platform is composed of sterilizable materials.

17. A catheter handle of a robotic catheter system, the catheter handle comprising:
a plurality of knobs configured to control a movement, an orientation, and/or a position of a distal end of the robotic catheter system;
a plurality of auxiliary gears connected to the plurality of knobs, the plurality of auxiliary gears configured to interface with a set of external gears on a base section; and
one or more magnetic contact points configured to magnetically secure the catheter handle to a mounting platform of the base section.

18. The catheter handle of claim 17,
wherein a first knob of the plurality of knobs is responsible for controlling a rotation of the robotic catheter system, wherein a second knob and third knob of the plurality of knobs are used for directional control.

19. The catheter handle of claim 17, wherein the one or more magnetic contact points are provided by magnetic tape on the catheter handle.

20. The catheter handle of claim 17, wherein the one or more magnetic contact points are provided by electromagnets embedded in the mounting platform.
